(19) United States
(12) Patent Application Publication
(10) Pub. No.: US 2011/0308541 A1
(43) Pub. Date: Dec. 22, 2011

(54) HAIR TREATMENT COMPOSITION

(76) Inventors: Wi-Soon Chia, Shanghai (CN); Artit Kijchotipaisarn, Bangkok (TH)

(21) Appl. No.: 13/146,647

(22) PCT Filed: Jan. 20, 2010
(86) PCT No.: PCT/EP10/50651

§ 371 Date: Sep. 8, 2011

(30) Foreign Application Priority Data
Feb. 4, 2009 (EP) 09152017.1

(51) Int. Cl. A61K 8/891; A61Q 5/06

(52) U.S. Cl. 132/207

(57) ABSTRACT

A method of styling hair comprising the steps of i) applying to the hair a hair treatment cream composition comprising: a) a blend of MQ resin and polydiorganosiloxane; and b) hydrophobically modified cellulose ether. ii) combing and/or styling the hair.

This invention relates to hair treatment compositions and to their use in the treatment of hair.

WO 2007/051505 (Unilever) describes pressure sensitive adhesives in leave on styling compositions.

The present invention relates to compositions that can be used to style hair yet enhance the ease of combing.

Accordingly, this invention provides a method of styling hair comprising the step of i) applying to the hair a hair treatment cream comprising:

a) a blend of MQ resin and polydiorganosiloxane; and
b) hydrophobically modified cellulose ether.

Unless specified otherwise, all wt % values quoted hereinafter are percentages by weight based on total weight of the hair treatment composition.

Compositions of the invention comprise a blend of MQ silicone resin and polydiorganosiloxane. Preferably such materials act as pressure sensitive adhesives. The term “pressure sensitive adhesive” (PSA) refers to materials that are permanently tacky at room temperature and able to develop measurable adhesion to a surface simply upon contact or by the application of a light pressure. Generally they do not require heat. No chemical reaction takes place between the adhesive and the adherent, no curing of the adhesive is necessary and no solvent is required to be lost during the adhesion process.

A preferred blend comprises (a) 40 to 70 parts by weight of at least one silicone copolymer resin and (b) 30 to 60 parts by weight of at least one polydiorganosiloxane.

A preferred MQ resin is (Me₃SiO_{1/2})ₓ(SiO_{4/2}) where 1<X<3, more preferably 0.5<x<1.5.

The described blends are preferably solvated. Examples of solvents are esters and alcohols, preferred solvents are silicones and hydrocarbon or mixtures thereof. A preferred solvent is cyclopentasiloxane.

It is preferable if the solvent for the blend is present at levels of at least 50 wt % of the total level of blend and solvent system, more preferably from 40 to 70 wt %, most preferably from 50 to 60 wt %.

The level of solvated blend is preferably from 0.001 to 3 wt % of the total composition, more preferably from 0.01 to 2 wt %, most preferably from 0.1 to 1 wt % of the total composition.

The composition of the invention comprises a hydrophobically modified cellulose ether as a thickening agent.

The hydrophobically modified cellulose ethers useful herein are preferably nonionic polymers. The hydrophobically modified cellulose ethers useful herein comprise a hydrophilic cellulose backbone and a hydrophobic substitution group.

The hydrophilic cellulose backbone has a sufficient degree of nonionic substitution to cellulose to be water soluble. Such hydrophilic cellulose backbone is selected from the group consisting of methyl cellulose, hydroxymethyl cellulose, hydroxyethyl cellulose, hydroxyethyl ethylcellulose, hydroxypropyl cellulose, hydroxypropyl methylcellulose, and mixtures thereof. The amount of nonionic substitution is not critical, so long as there is an amount sufficient to assure that the hydrophilic cellulose backbone is water soluble. The hydrophilic cellulose backbone has a molecular weight of about less than 800,000, preferably from about 20,000 to about 700,000, or from about 75 D. P. to about 2500 D. P. Further, where a high viscosity building effect is not desirable, a lower molecular weight cellulose backbone is preferred. One of the preferred hydrophilic cellulose backbone is hydroxyethyl cellulose having a molecular weight of from about 50,000 to about 700,000. Hydroxyethyl cellulose of this molecular weight is known to be one of the most hydrophilic of the materials contemplated. Thus, hydroxyethyl cellulose can be modified to a greater extent than other hydrophilic cellulose backbones.

The hydrophilic cellulose backbone is further substituted with a hydrophobic substitution group via an ether linkage to render the hydrophobically modified cellulose ether to have less than 1% water solubility, preferably less than 0.2% water solubility. The hydrophobic substitution group is selected from a straight or branched chain alkyl group of from about 10 to about 22 carbons; wherein the ratio of the hydrophilic groups in the hydrophilic cellulose backbone to the hydrophobic substitution group being from about 2:1 to about 1000:1, preferably from about 10:1 1 to about 100:1.

Commercially available hydrophobically modified cellulose ethers useful herein include: cetyl hydroxyethylcellulose having tradenames NATROSOL PLUS 330CS and POLYSURF 67, both available from Aqualon Company, Delaware, USA, having cetyl group substitution of about 0.4% to about 0.65% by weight of the entire polymer.

The hydroxyethyl cellulose or derivatives thereof are preferably present at levels from 0.01 to 3wt % of the total composition, more preferably from 0.01 to 0.1 wt %, most preferably at levels below 0.1 wt %.

The ratio of blend of MQ resin/polydiorganosiloxane
a) to hydrophobically modified cellulose
b) is preferably from 1:1 to 10:1, more preferably 2:1 to 12:1, most preferably from 5:1 to 10:1.

The compositions of the invention may further comprise from 0.001% to 10% by weight of a hair styling polymer.

More preferred amounts of hair styling polymer in the compositions of the invention are from 0.1% to 5% by weight of the composition, even more preferably from 0.5% to 3% by weight. However it is highly preferable if additional hair styling polymers are not present or present in levels below 0.01 wt % of the total composition.

Hair styling polymers are well known. Suitable hair styling polymers include commercially available polymers that contain moieties that render the polymers cationic, anionic, amphoteric or nonionic in nature. Suitable hair styling polymers include, for example, block and graft copolymers. The polymers may be synthetic or naturally derived.

The compositions of the invention may comprise surfactant. The surfactants which are suitable for use in the compositions of the invention may be nonionic, cationic, anionic, zwitterionic or a mixture of such surfactants depending on the product form.

It is preferable if the total composition comprises below 3 wt % of anionic and non-ionic surfactant, more preferably below 1 wt %.

Compositions of the present invention can also include water. The water will typically be present in amounts ranging from 30% to 98%, preferably from 50% to 95% by weight.

Hair conditioning agents such as hydrocarbons, esters, silicone fluids, and cationic materials may be included in the compositions of the invention. Hair conditioning agents may typically be present in compositions of the invention in amounts of from 0.001% to 10% by weight, preferably 0.1% to 3% by weight. Hair conditioning agents may be single compounds or mixtures of two or more compounds from the same class or different general classes.

Hair conditioning agents may be included in any of the compositions of the invention, regardless of whether they contain a hair styling polymer. In one embodiment of the invention, the compositions (such as aerosol mousse formulations, for example) comprise a hair conditioning agent and are substantially free of hair styling polymer.

Suitable hydrocarbons can be either straight or branched chain and can contain from about 10 to about 16, preferably from about 12 to about 16 carbon atoms. Examples of suitable hydrocarbons are decane, dodecane, tetradecane, tridecane, and mixtures thereof.

Suitable oily or fatty materials are selected from hydrocarbon oils, fatty esters and mixtures thereof.

Straight chain hydrocarbon oils will preferably contain from about 12 to about 30 carbon atoms. Also suitable are polymeric hydrocarbons of alkenyl monomers, such as C₂-C₆ alkenyl monomers.

Specific examples of suitable hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, and mixtures thereof. Branched-chain isomers of these compounds, as well as of higher chain length hydrocarbons, can also be used.

Suitable fatty esters are characterised by having at least 10 carbon atoms, and include esters with hydrocarbyl chains derived from fatty acids or alcohols. Monocarboxylic acid esters include esters of alcohols and/or acids of the formula R′COOR in which R′ and R independently denote alkyl or alkenyl radicals and the sum of carbon atoms in R′ and R is at least 10, preferably at least 20. Di- and trialkyl and alkenyl esters of carboxylic acids can also be used.

Particularly preferred fatty esters are mono-, di- and triglycerides, more specifically the mono-, di-, and tri-esters of glycerol and long chain carboxylic acids such as C₁-C₂₂ carboxylic acids. Preferred materials include cocoa butter, palm stearin, sunflower oil, soyabean oil and coconut oil.

The oily/fatty material is suitably present at a level of from 0.05 to 10, preferably from 0.2 to 5, more preferably from about 0.5 to 3 wt %.

Examples of suitable silicone conditioning agents useful herein can include either cyclic or linear polydimethylsiloxanes, phenyl and alkyl phenyl silicones, and silicone copolyols. Cationic conditioning agents useful herein can include quaternary ammonium salts or the salts of fatty amines, such as cetyl ammonium chloride, for example.

Compositions according to the invention may, optionally, comprise from 0.1% to 10% by weight of a volatile silicone as the hair conditioning agent. Volatile silicones are well known in the art and are commercially available and include, for example linear and cyclic compounds. Volatile silicone oils are preferably linear or cyclic polydimethylsiloxanes containing from about three to about nine silicon atoms.

Compositions of the invention may comprise a further silicone, preferably in the form of an emulsion.

Suitable silicone emulsions include those formed from silicones such as polydiorganosiloxanes, in particular polydimethylsiloxanes which have the CTFA designation dimethicone, polydimethyl siloxanes having hydroxyl end groups which have the CTFA designation dimethiconol, and amino-functional polydimethyl siloxanes which have the CTFA designation amodimethicone.

The emulsion droplets may typically have a Sauter mean droplet diameter (D_{3,2}) in the composition of the invention ranging from 0.01 to 20 micrometer, more preferably from 0.2 to 10 micrometer.

A suitable method for measuring the Sauter mean droplet diameter (D_{3,2}) is by laser light scattering using an instrument such as a Malvern Mastersizer.

Suitable silicone emulsions for use in compositions of the invention are available from suppliers of silicones such as Dow Corning and GE Silicones. The use of such pre-formed silicone emulsions is preferred for ease of processing and control of silicone particle size. Such pre-formed silicone emulsions will typically additionally comprise a suitable emulsifier such as an anionic or nonionic emulsifier, or mixture thereof, and may be prepared by a chemical emulsification process such as emulsion polymerisation, or by mechanical emulsification using a high shear mixer. Pre-formed silicone emulsions having a Sauter mean droplet diameter (D_{3,2}) of less than 0.15 micrometers are generally termed microemulsions.

Examples of suitable pre-formed silicone emulsions include emulsions DC2-1766, DC2-1784, DC-1785, DC-1786, DC-1788 and microemulsions DC2-1865 and DC2-1870, all available from Dow Corning. These are all emulsions/microemulsions of dimethiconol. Also suitable are amodimethicone emulsions such as DC939, DC7134 (from Dow Corning) and SME253 (from GE Silicones).

Mixtures of any of the above described silicone emulsions may also be used.

Silicone will generally be present in a composition of the invention at levels of from 0.05 to 10%, preferably 0.05 to 5%, more preferably from 0.5 to 2% by total weight of silicone based on the total weight of the composition.

The compositions of the invention may optionally comprise cationic surfactants, used singly or in admixture.

Compositions according to the invention comprise one or more cationic surfactants which are cosmetically acceptable and suitable for topical application to the hair.

Suitable cationic surfactants for use in compositions of the invention contain amino or quaternary ammonium hydrophilic moieties which are positively charged when dissolved in the composition.

Suitable quaternary ammonium cationic surfactants correspond to the following general formula (I):

[N(R¹)(R²)(R³)(R⁴)]⁺ (X)⁻ (I)

in which R¹, R², R³, and R⁴ are each independently selected from (a) an aliphatic group of from 1 to 22 carbon atoms, or (b) an aromatic, alkoxy, polyoxyalkylene, alkylamido, hydroxyalkyl, aryl or alkylaryl group having up to 22 carbon atoms; and X is a salt-forming anion such as those selected from halogen, (e.g. chloride, bromide), acetate, citrate, lactate, glycolate, phosphate nitrate, sulphate, and alkylsulphate radicals.

The aliphatic groups can contain, in addition to carbon and hydrogen atoms, ether linkages, and other groups such as amino groups. The longer chain aliphatic groups, e.g., those of about 12 carbons, or higher, can be saturated or unsaturated.

In a suitable class of cationic surfactant of general formula (I), R¹ and R² are each independently selected from C₁₆ to C₂₂ hydrocarbyl chains comprising at least one ester linkage in both R¹ and R², and R³ and R⁴ are each independently selected from CH₃ and CH₂CH₂OH.

In another suitable class of cationic surfactant of general formula (I), R¹ and R² are each independently selected from C₁₆ to C₂₂ saturated or unsaturated, preferably saturated, chains, and R³ and R⁴ are each independently selected from CH₃ and CH₂CH₂OH, preferably CH₃.

In a preferred class of cationic surfactant of general formula (I), R¹ is a C₁₆ to C₂₂ alkyl chain and R², R³ and R⁴ are each independently selected from CH₃ and CH₂CH₂OH, preferably CH₃.

Specific examples of suitable quaternary ammonium cationic surfactants of general formula (I) are cetyltrimethylammonium chloride, behenyltrimethylammonium chloride (BTAC), cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, cocotrimethylammonium chloride, dipalmitoylethyldimethylammonium chloride, PEG-2 oleylammonium chloride and salts of these, where the chloride is replaced by halogen, (e.g., bromide), acetate, citrate, lactate, glycolate, phosphate nitrate, sulphate, or alkylsulphate. Particularly preferred quaternary ammonium cationic surfactants for use in the invention are cetyltrimethylammonium chloride, available commercially, for example as GENAMIN CTAC, ex Hoechst Celanese and Arquad 16/29 supplied by Akzo Nobel, and behenyltrimethylammonium chloride (BTAC) such as Genamin KDM-P supplied by Clariant.

Mixtures of any of the foregoing materials may also be suitable.

Salts of primary, secondary, and tertiary fatty amines are also suitable cationic surfactants for use in the invention. The alkyl groups of such amines preferably have from about 12 to about 22 carbon atoms, and can be substituted or unsubstituted. These amines are typically used in combination with an acid to provide the cationic species.

A preferred class of amine corresponds to the following general formula (II):

R¹—C(O)—N(H)—R²—N(R³)(R⁴) (II)

in which R¹ is a fatty acid chain containing from 12 to 22 carbon atoms, R² is an alkylene group containing from one to four carbon atoms, and R³ and R⁴ are, independently, an alkyl group having from one to four carbon atoms.

Specific examples of suitable materials of general formula (II) are stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, and diethylaminoethylstearamide.

Also useful are dimethylstearamine, dimethylsoyamine, soyamine, myristylamine, tridecylamine, ethylstearylamine, N-tallowpropane diamine, ethoxylated (with 5 moles of ethylene oxide) stearylamine, dihydroxyethylstearylamine, and arachidyl behenylamine.

Particularly preferred is stearamidopropyldimethylamine.

Mixtures of any of the foregoing materials may also be suitable.

The acid used to provide the cationic species can be any organic acid or mineral acid of sufficient acid strength to neutralise a free amine nitrogen. Such acids include hydrochloric acid, sulphuric acid, nitric acid, phosphoric acid, lactic acid, citric acid, tartaric acid, acetic acid, gluconic acid, glycolic acid and propionic acid, or combinations thereof. In general, a sufficient amount of acid is added to neutralise the amidoamine compound and to adjust the final pH of the composition to within a range of from about 2.5 to about 6, preferably in a pH range of from about 3 to about 5. The molar ratio of protonatable amine groups to H⁺ from the acid is preferably from about 1:0.3 to 1:1.2, and more preferably from about 1:0.5 to about 1:1.1.

Mixtures of any of the above-described cationic surfactants may also be suitable.

In the composition of the invention, the level of cationic surfactant preferably ranges from 0.1 to 10%, more preferably 0.2 to 5%, most preferably 0.25 to 4% by total weight of cationic surfactant based on the total weight of the composition.

Conditioner compositions of the invention may additionally comprise a fatty alcohol material. The combined use of fatty alcohol materials and cationic surfactants in conditioning compositions is believed to be especially advantageous, because this leads to the formation of a lamellar phase, in which the cationic surfactant is dispersed.

By “fatty alcohol material” is meant a fatty alcohol, an alkoxylated fatty alcohol, or a mixture thereof.

Representative fatty alcohols comprise from 8 to 22 carbon atoms, more preferably 16 to 20. Examples of suitable fatty

Compositions of the present invention are preferably formulated into leave in formulations.

It is preferred if the products are left on hair after application and not immediately washed off (within one hour of application, more preferably the products are left for at least 4 hours on the hair, most preferably the products are only applied when the hair is to be styled and not removed until the hair needs restyling which is usually once or twice a day).

The compositions of the invention may take a variety of forms, including, for example, mousses, gels, lotions, creams, sprays and tonics. These product forms are well known in the art.

The preferred product is a cream.

The cream may include a further structurant or thickener, typically at a level from 0.1% to 10%, preferably 0.5% to 3% by weight based on total weight.

Examples of suitable structurants or thickeners are polymeric thickeners such as carboxyvinyl polymers. A carboxyvinyl polymer is an interpolymer of a monomeric mixture comprising a monomeric olefinically unsaturated carboxylic acid, and from about 0.01% to about 10% by weight of the total monomers of a polyether of a polyhydric alcohol. Carboxyvinyl polymers are substantially insoluble in liquid, volatile organic hydrocarbons and are dimensionally stable on exposure to air. Suitably the molecular weight of the carboxyvinyl polymer is at least 750,000, preferably at least 1,250,000, most preferably at least 3,000,000. Preferred carboxyvinyl polymers are copolymers of acrylic acid cross-linked with allylsucrose or allylpentaerythritol as described in U.S. Pat. No. 2,798,053. These polymers are provided by B.F.Goodrich Company as, for example, CARBOPOL 934, 940, 941 and 980. Other materials that can also be used as structurants or thickeners include those that can impart a gel-like viscosity to the composition, such as water soluble or colloidally water soluble polymers like cellulose ethers (e.g. methylcellulose, hydroxyethylcellulose, hydroxypropylmethylcellulose and carboxymethylcellulose), guar gum, sodium alginate, gum arabic, xanthan gum, polyvinyl alcohol, polyvinyl pyrrolidone, hydroxypropyl guar gum, starch and starch derivatives, and other thickeners, viscosity modifiers, gelling agents, etc. It is also possible to use inorganic thickeners such as bentonite or laponite clays.

The hair compositions of the invention can contain a variety of non-essential, optional components suitable for rendering the compositions more aesthetically acceptable or to aid use, including discharge from the container, of the product. Such conventional optional ingredients are well known to those skilled in the art, e.g. preservatives such as benzyl alcohol, methyl paraben, propyl paraben and imidazolidinyl urea, fatty alcohols such as cetearyl alcohol, cetyl alcohol and stearyl alcohol, pH adjusting agents such as citric acid, succinic acid, sodium hydroxide and triethanolamine, colouring agents such as any of the FD&C or D&C dyes, perfume oils, chelating agents such as ethylenediamine tetraacetic acid, and polymer plasticising agents such as glycerin and propylene glycol. The invention will now be further illustrated by the following, non-limiting Examples.

Examples of the invention are illustrated by a number, comparative examples are illustrated by a letter.

| | Example A | Example 1 | Example 2 |
|---|---|---|---|
| INCI Name | % w/w | % w/w | % w/w |
| Dimethiconol | 0.70 | 0.70 | 0.70 |
| Dimethicone (and) | 0.30 | 0.30 | 0.30 |
| Amodimethicone | | | |
| MₓQ resin (0.5 < X < 1.5) | — | 0.20 | 0.40 |
| with PDMS in D5 | | | |
| Stearamidopropyl Dimethylamine | 1.00 | 1.00 | 1.00 |
| Cetearyl Alcohol (100%) | 3.00 | 3.00 | 3.00 |
| Cetyl Hydroxyethyl cellulose | 0.05 | 0.05 | 0.05 |
| Glycerin | 2.00 | 2.00 | 2.00 |
| Isopropyl palmitate | 4.00 | 4.00 | 4.00 |
| Water | to 100 | to 100 | to 100 |

Trained panellists assessed hair switches treated with 5 g of product to the hair.

| | Nomalised scores | | |
|---|---|---|---|
| | Example A | Example 1 | Example 2 |
| Left the hair feeling smooth | 22 | 41 | 45 |
| Left the hair fluffy | 57 | 27 | 24 |
| Hair was easy to comb | 25 | 32 | 51 |

The results show a significant difference between the comparative example and the examples of the invention.

Examples of the invention were significantly easier to comb, felt smoother and were less fluffy.

1. A method of styling hair comprising the steps of i) applying to the hair a hair treatment cream composition comprising:
a) a blend of MQ resin and polydiorganosiloxanes which is a pressure sensitive adhesive; and
b) hydrophobically modified cellulose ether; and
ii) combing and/or styling the hair.

2. A method according to claim 1 in which the MQ resin has the formula:
(Me₃SiO_{1/2})ₓ(SiO_{4/2})
where 1<X<3.

3. A method according to claim 1 in which the blend of MQ resin and polydiorganosiloxane is solvated.

4. A method according to claim 1 in which the hydrophobically modified cellulose is ether cetyl hydroxyethylcellulose.

5. A method according to claim 1 in which the composition further comprises a hair conditioning agent.
